(19) United States
(12) Patent Application Publication
(10) Pub. No.: US 2009/0060304 A1
(43) Pub. Date: Mar. 5, 2009

(54) DERMATOLOGY INFORMATION

(76) Inventors: Joseph V. Gulfo, New York, NY (US); Nikolai Kabelev, Irvington, NY (US); Dina Gutkowicz-Krusin, Princeton, NJ (US)

Correspondence Address: FISH & RICHARDSON PC, P.O. BOX 1022, MINNEAPOLIS, MN, 55440-1022, US

(21) Appl. No.: 12/204,247

(22) Filed: Sep. 4, 2008

Related U.S. Application Data
(60) Provisional application No. 60/969,898, filed on Sep. 4, 2007.

(51) Int. Cl. A61B 6/00; G06K 9/00

(52) U.S. Cl. 382/128; 600/476

(57) ABSTRACT

Among other things, in connection with scanning a lesion on the skin of a subject, the location of the lesion on the subject is automatically determined. The scan and the location are stored on a portable memory device. And the memory device is included in a physical patient record of the subject for later use.

### DERMATOLOGY INFORMATION

Under 35 U.S.C. §119(e)(1), this application claims the benefit of the filing date of U.S. provisional patent application 60/969,898, entitled “DERMATOLOGY INFORMATION,” filed on Sep. 4, 2007, the entire contents of which are incorporated here by reference.

### BACKGROUND

This description relates to dermatology information.

Dermatology information can include digital information generated by optical scanning of lesions, for example, digital images of the lesions, and metadata associated with the scans such as the date, time, and place in which the scanning was done, demographic information about the patient, and information about the location of the lesion on the body. Typically, the dermatology information is recorded by the doctor or other clinician on paper or temporarily through a user interface of a computer and then onto paper. Often the information is stored on paper in a physical patient file for later use and reference. Some kinds of dermatology information are recorded and stored electronically and can be sent to other users and places through a communication network, such as the Internet.

The location of a lesion can be indicated by the doctor using marks on a drawing or picture of the body and in other ways, for example, providing input on a touch-sensitive screen that is depicting a body map. The doctor can use a previously acquired picture of a mole on a patient's body for comparison with the mole as currently observed to aid in diagnosing whether the mole is benign or malignant. This procedure is sometimes called mole mapping.

### SUMMARY

In general, in an aspect, in connection with scanning a lesion on the skin of a subject, the location of the lesion on the subject is automatically determined. The scan and the location are stored on a portable memory device. And the memory device is included in a physical patient record of the subject for later use.

Implementations may include one or more of the following features. The scanning includes deriving image information corresponding to the lesion. The scanning is performed in a manner that reduces the effects of ambient light on image information generated by the scan. The location of a suspicious pigmented lesion that has been evaluated is automatically captured by hardware and software by triangulating the location of a suspicious pigmented lesion that has been evaluated. The triangulation includes sending signals between transceivers located in fixed locations relative to the subject and a device located on or near and at a fixed location relative to the subject. The portable memory device is flash memory. An orientation of the lesion on the subject is automatically determined. The lesion is scanned again at a later time. The lesion includes a pigmented skin lesion (e.g., a mole). Changes in the lesion represented by the scans of the lesion are automatically analyzed and reported to a clinician. An indication is received (e.g., from the clinician) that the lesion is of interest to be later scanned for changes. The fact that the lesion is of interest is identified to a clinician at a later time, for example by a graphical indication on a depiction of the subject's body.

In general, in an aspect, in the presence of ambient light having a first set of characteristics, first image information about a lesion on the skin of a subject is captured using a scanner that screens the ambient light from the lesion. Later, in the presence of ambient light having a second set of characteristics, second image information about the lesion is captured using the scanner. By machine, the first image information and the second image information is analyzed to infer changes in characteristics of the lesion. The machine analyzes changes in at least one of the size, color, or shape of the lesion. The results of the analysis are displayed to a clinician. The image information is in an RGB format. The machine may also be configured to analyze changes in the lesion based on multi-spectral properties of the lesion that may not be visible in an RGB image.

In general, in an aspect, image scans of a pigmented skin lesion of interest on a patient are received at two different times with respect to which at least one of the color, size, shape, or non-visible multi-spectral characteristics of the pigmented skin lesion may have changed. An analysis of the changes of the pigmented skin lesion is performed by machine. The result of the analysis is reported to a clinician through a user interface.

A wide variety of other dermatology information can also be captured, saved, and used for a wide variety of purposes, for example, information regarding computer-assisted analysis of multi-spectral information about a pigmented skin lesion. Information in this instance includes, but is not limited to, an automated recommendation as to whether a biopsy to rule out melanoma is needed, or whether a clinician may desire to track a suspicious pigmented lesion over time at subsequent examinations, for example, mole mapping.

In general, in an aspect, two images of a pigmented skin lesion acquired at two different times are stored on a portable memory device. Information about the location of the pigmented skin lesion on the patient is stored on the portable memory device. An indication that that pigmented skin lesion is of interest is stored on the portable memory device. The images, location information, and indication of interest information are displayed to a clinician in connection with a pigmented skin lesion mapping procedure.

These and other features and aspects, and combinations of them, can be expressed as methods, apparatus, systems, program products, as means for performing a function, and in other ways.

Other advantages and features will become apparent from the description and the claims.

### DESCRIPTION

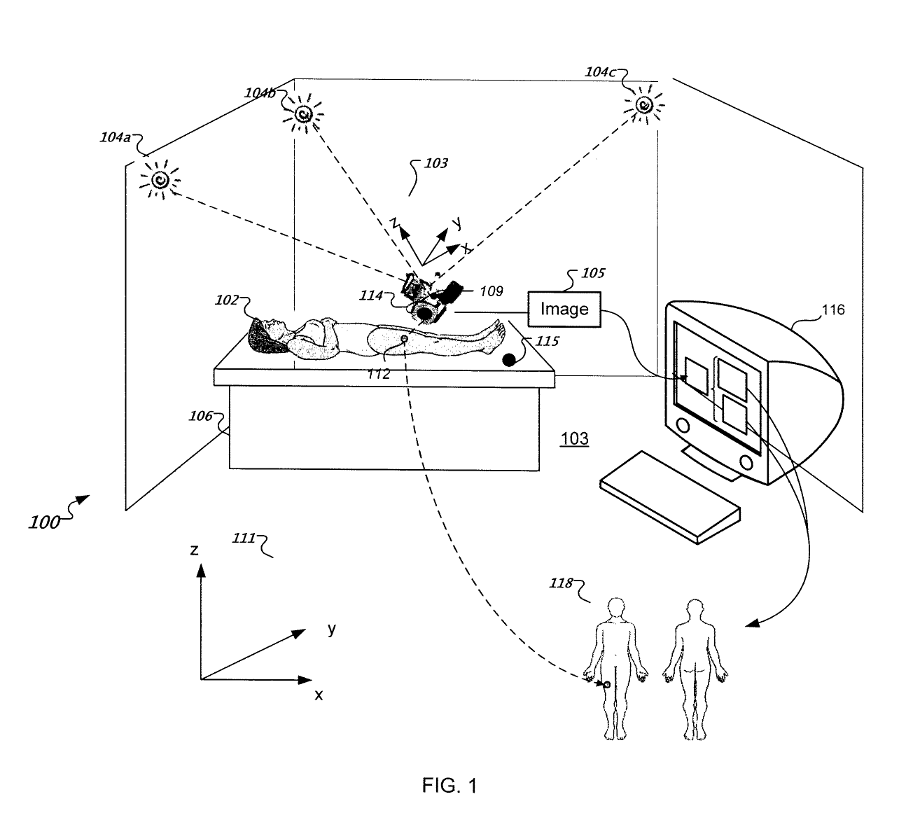

FIG. 1 is a perspective view of an examination room.

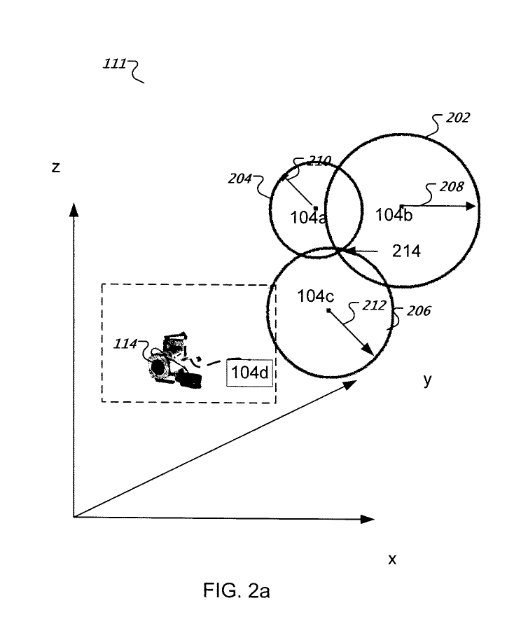

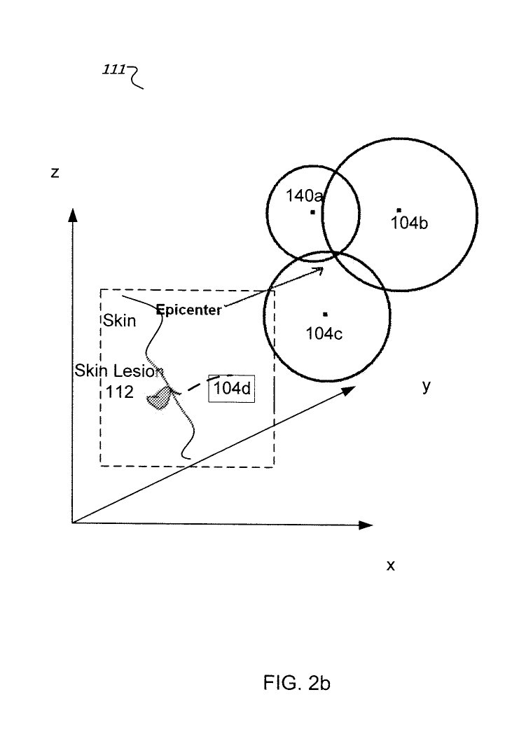

FIGS. 2a and 2b are diagrams of a triangulation method.

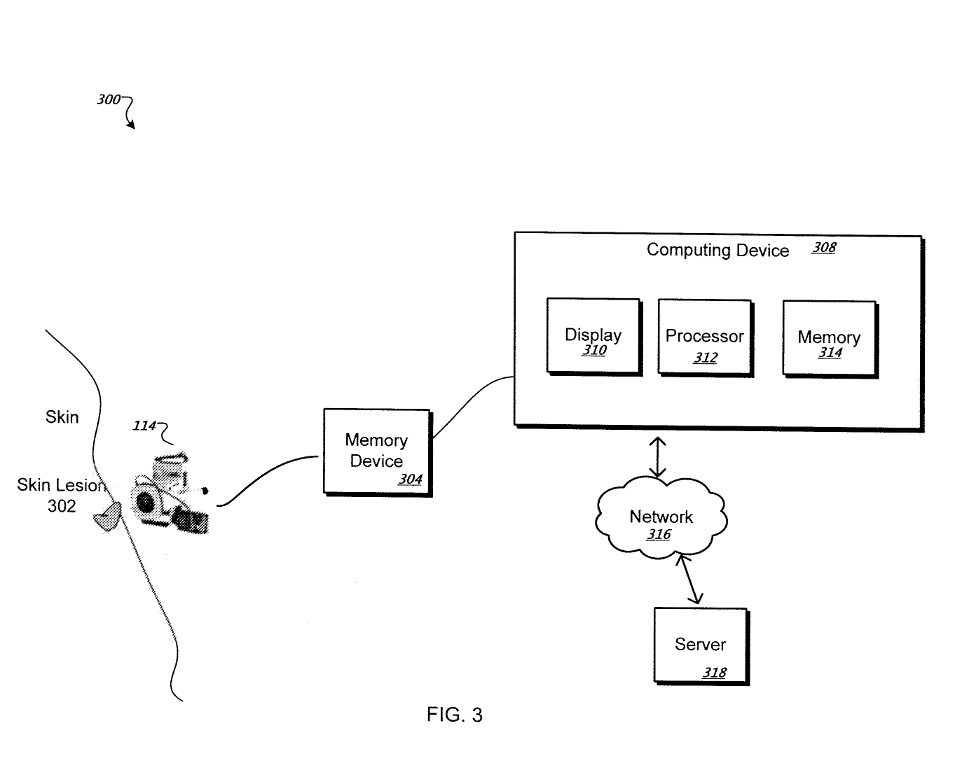

FIG. 3 is a block diagram.

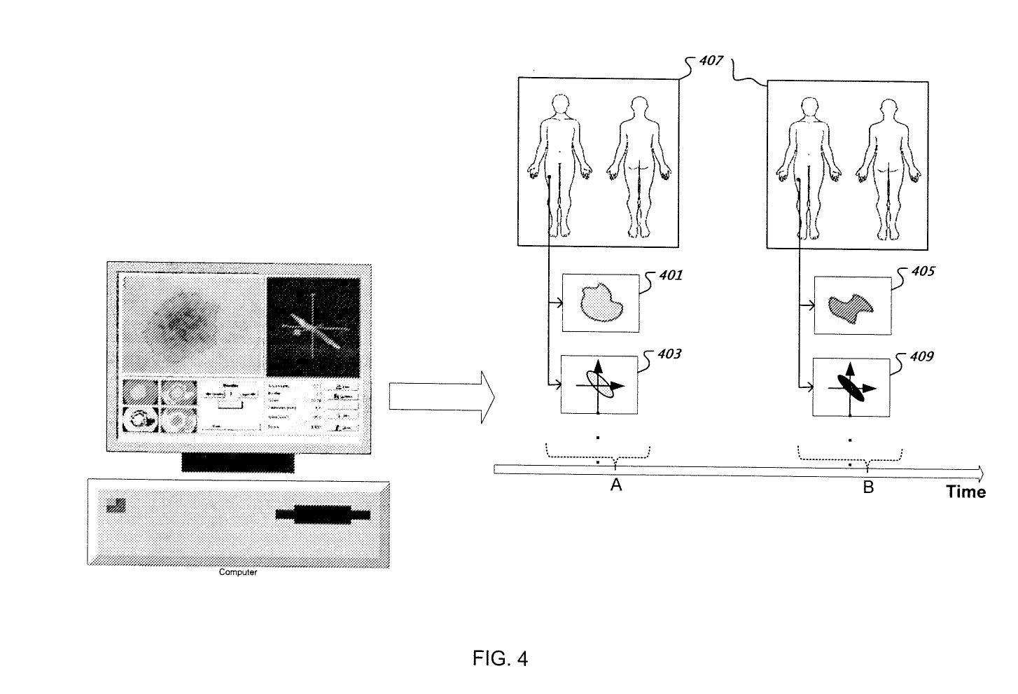

FIG. 4 is a time chart.

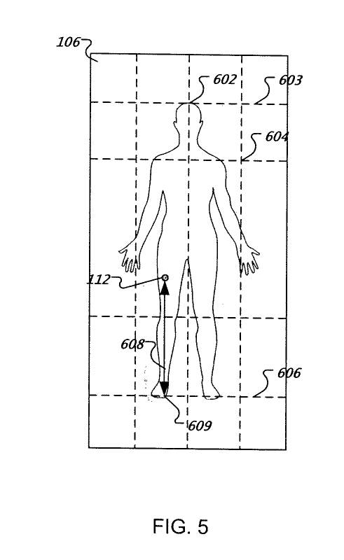

FIG. 5 is a body diagram.

Traditionally, a medical professional often relies on the appearance of a patient's skin and an evaluation of macroscopic features seen on the skin in diagnosing a skin lesion. In some cases, such as mapping of a mole or other pigmented skin lesion, changes in the size, shape, color and other characteristics over time can indicate whether the mole is benign or malignant.

In the case of mole mapping, as an example, each time the clinician observes the mole, he may take notes that record an evaluation of the mole, its location, size, shape, color, and other characteristics. The notes and one or more photographs of the mole can be kept in the patient's record for use in determining changes in the condition of the mole in one or more subsequent patient visits. The observed changes can be useful in diagnosing the mole.

The accuracy of a diagnosis based on mole mapping depends on the clinician's experience and visual acuity. Moles that are benign usually require no treatment. Some atypical moles, however, may develop into melanoma. Analyzing changes in the pigmented skin lesion sometimes referred to as mole mapping. In general, when we refer to moles we mean to include any kind of pigmented skin lesion. Although we often use moles and mole mapping as one example in our discussion, the techniques described here can be applied to any kind of skin lesion and used for purposes other than observing the changes of a particular lesion over time.

Here we describe a system that enables a clinician to collect and store information about a skin lesion, including digital images and metadata, for example, and to identify and store the location of a suspicious lesion automatically for use in later examinations or for other purposes. When we refer to a clinician, we mean to include any medical professional, assistant, or other user or operator of the system.

The lesion information can be stored on a portable computer memory device such as flash memory and held as part of a physical or electronic patient record, making it easier to track changes in lesions (for example suspicious lesions) over time. By determining the location of the lesion on the body optically or electromagnetically and in some cases automatically, the location identification can be more accurate and can be acquired without requiring time and effort on the part of the clinician. Reproducible digital images of the scanned lesions can be generated and stored automatically and associated with the location information. The stored images, metadata, and locations, as part of the patient's medical information, can be used later in a wide variety of ways that go beyond the uses that can be made of simple markings, notes, or measurements typically made by clinicians on cartoon drawings or pictures of the body or parts of the body.

As shown in FIG. 1, in some implementations of a skin characteristics acquisition and analysis system 100, a patient 102 in an examination room 103 in a healthcare environment (e.g., a hospital or a dermatologist's office) lies on an examination table 106 while a skin lesion 112 is inspected and scanned. During a typical examination more than one lesion may be inspected and scanned and our description here applies to each of the inspections and scans.

The scan of the lesion is taken using a digital scanner 114 that can be any of a wide variety of digital still cameras, video cameras, camcorders, scanners, or other electrical, optical, acoustical, or chemical image recording device that is capable of producing reliable, consistent, and reproducible images (two-dimensional or three-dimensional) of the skin lesion 112 may be used. Typically the image information is in the form of pixels that each represents an intensity and color (for example, in a full-color gamut or as intensities on multiple spectral scans of the lesion) of a tiny spot on the lesion. It is desirable to use an image capture device that can provide reproducible identical image results at different times and different ambient light conditions so that light conditions will not affect the comparison of successive images of a lesion taken at different times and different places and under different lighting conditions. In some examples, the scanner is a MelaFind® scanner of the kind developed by Electro-Optical Sciences of Irvington, N.Y., and aspects of which are described for example, in U.S. Pat. No. 6,081,612, filed Feb. 27, 1998, U.S. Pat. No. 6,208,749, filed Feb. 27, 1998, U.S. Pat. No. 6,307,957, filed Jun. 27, 2000, U.S. Pat. No. 6,563,616, filed Feb. 21, 1999, U.S. Pat. No. 6,626,558, filed Aug. 31, 2001, U.S. Pat. No. 6,657,798, filed Feb. 10, 2003, U.S. Pat. No. 6,710,947, filed Feb. 10, 2003, U.S. Pat. No. 7,102,672, filed Feb. 8, 2002, and U.S. Pat. No. 7,127,094, filed Jan. 2, 2003, and U.S. patent application Ser. Nos. 11/500,197, filed Aug. 7, 2006, 11/681,345, filed Mar. 2, 2007, 11/761,816, filed Jun. 12, 2007, and 11/956,918, filed Dec. 14, 2007, each incorporated here by reference. Among other advantages, the MelaFind scanner makes contact with the lesion during scanning, shielding it from ambient light, and provides carefully controlled lighting of the lesion in multiple spectral bands, making the image information acquired during the scans repeatable and consistent.

Note that, in FIG. 1, the scanner is shown held above the lesion. When the scanner is a MelaFind scanner, for example, the scanner is held down on the patient's skin, and the lesion is in contact with a light controlled chamber of the scanner and not visible to the clinician.

In some examples, image(s) of the skin lesion 112 captured by a digital camera (e.g., the digital scanner 114 in FIG. 1) may be displayed and processed in real time on a graphical user interface (GUI) on the computer 116. The images can also be stored in, for example, in a portable computer memory device (e.g., a memory device 304 in FIG. 3) for displaying, processing, and analyzing at a later time at a different location or at the same location. Additional information about an example of such a memory device and its use in lesion scanning may be found in U.S. patent application Ser. No. 11/761,816, filed Jun. 12, 2007, all of which is incorporated here by reference.

The image information 105 is acquired (we sometimes call this taking the image) when the clinician pulls a trigger on the scanner or presses a shutter release on a camera, for example, and is then delivered to a computer 116 where it can be stored, processed, and retrieved, for example. The system 100 is arranged to track the location of a reference point 109 on the scanner and an orientation 113 of the scanner 114 relative to the reference point 109 and to acquire and store the three-dimensional location and orientation at the moment when the clinician takes the image. The location of the lesion within the image-taking chamber of the scanner and the location and direction of the reference point 109 relative to lesion are known by virtue of the design of the scanner. Accordingly, the location of the lesion and the orientation of the lesion on the surface of the patient's skin can be determined automatically at the time when the picture is taken relative to a fixed reference location 115 in the examination room or even on the patient.

The orientation of the scanner can be determined using an inertial sensor or other orientation detector (not shown) within the scanner or by other techniques.

The location of the scanner relative to the reference location 115 and then the three-dimensional location and orientation of the reference point and the lesion can be determined using a triangulation technique similar to the one used in satellite-based geographical positioning systems (GPS). Then the relative location and orientation of the lesion on the patient's body can be derived automatically and stored along with the scan and metadata.

In some examples of triangulation (there are a wide variety of other approaches that can be used), three transceivers 104a, 104b, 104c are mounted at known locations of the examination room 103, e.g., at selected room corners or fixed positions on the side walls of the examination room 103. The geographic relationship of the three transceivers 104a, 104b, 104c with respect to each other and other objects in the examination room 103 (e.g., the examination table 106, the digital scanner 114, and the reference location 115) are known in the three-dimensional space 111.

As shown in FIG. 2a, the digital scanner 114 may include a transmitter 104d at the reference point 109. In cases in which the scanner is a digital camera held at a distance from the patient's skin, the transmitter 104d could be placed on or adjacent to the lesion 112 of the patient 102 during a scan (see FIG. 2b). In the former case, because the transmitter 104d is integrated with the digital scanner 114, the location of the lesion 112 can be easily acquired at the same time the image is taken.

To scan the lesion 112, the clinician adjusts the scanner 114 to frame a region including the lesion. A visual indication of the location of the scanner relative to the lesion may be provided on a view finder on the digital scanner 114 or on the display of the computer 116 or in other ways. Once the desired skin area has been framed, the clinician may simply push a button on the digital scanner 114 to take the image and the system simultaneously records location and orientation of the lesion 112.

In order to capture the location of the lesion on the patient, either the reference location 115 (e.g., a marker or transmitter the location of which can be tracked by the system) must be placed at a known location on the patient, such as a standard place on the ankle or wrist or waist or neck. Or the location and orientation of the patient relative to the table 106 and therefore to the reference transmitter at location 115 on the table must be carefully established when the patient lies on the table so that locations on the patient's body can be inferred with reasonable accuracy. Note that the location and orientation of the lesion on the patient's body, in some implementations, need not be any more accurate than a few centimeters, because the goal is only to enable the clinician to quickly find the lesion on a subsequent examination.

As shown in FIG. 2a, when the image is taken, the transmitter 104d can be triggered to broadcast in all directions an optical or radio frequency (RF) signal. Each of the triangulation transceivers 104a, 104b, 104c typically has a working range, and a reference distance, also known as standoff distance (provided by manufacturers). The standoff distance is the mid point in the working range. The working range permits measurement of distances either more or less the reference distance and is usually given as a +/−value. For example, a transceiver with a reference distance of 9″ and a working range of +/−4″ will have an effective total range of 8″ over which it can measure, the midpoint of that range being 9″ from the transceiver. This means that the distance being measured is between 5″ and 13″ from the transceiver. The strength of the signal from the transmitter is measured at each of the three transceivers 104a, 104b, 104c. The transmitter 104d can be inferred to lie on a sphere at a distance from each of the transceivers that corresponds to the signal strength detected.

Triangulation is used to find the intersection of the three spheres. In practice, three spheres can intersect at two points, at one point, or not at all depending on the locations of the three transceivers 104a, 104b, 104c relative to one another and relative to the transmitter 104d. By appropriate placing of the three transceivers 104a, 104b, 104c in the examination room 103, the possibility of there being no intersecting point can be eliminated for the space in which the patient lies. Furthermore, the selection of which of two intersecting points is the one of interest can be handled by inference to eventually determine spheres 202, 204 and 206 that intersect at a single point 214 (also referred to as the epicenter). The computer 116 in FIG. 1 runs algorithms to resolve the trigonometric relationships to translate the position of the epicenter (i.e., the location of the lesion 112) into the three-dimensional space 111, from which the location of the lesion (relative to the transmitter 104d) can be determined.

The triangulation transceivers 104a, 104b, 104c can use either visible or invisible light beams (or RF signals). Use of a visible beam can make the sensors easy to mount and aim at a target. Triangulation transceivers can operate with almost any type of light source.

In the case when the scanner is at a distance from the lesion when the image is taken (see FIG. 2b) and a light source of high intensity can create a small spot on the patient 102 skin surface, for example, a laser or solid-state laser diode. In particular, solid-state laser diodes may operate continuously, or they may be modulated or pulsed. Using a modulated laser can be useful in reducing the interference of ambient light by filtering the detector output at the modulation frequency or using lock-in amplifier technologies. Laser wavelength, or color, has no significant influence on triangulation performance, provided the sensor senses the wavelength. Using a visible wavelength laser, which is similar to a laser pointer with a focused point of light, can be useful for the clinician, because he or she can easily see the laser is on when a visible source is used. This also serves as a source of comfort for the user and a quick check for diagnostic purposes. Any light source that complies with the safety regulations of governmental agencies and can be used.

The transceivers 104a, 104b, 104c are controlled by the computer 116 through wired or wireless connections.

Referring to FIG. 5, in order to link the location of lesion 112 automatically in connection with an anatomical drawing 118 of the patient 102 generated or stored by the computer 116, a reference point 609 or a reference plane may be defined prior to each skin scan. For this purpose, the patient may be placed in a fixed, known positioned on the examination table 106. Then one or more known points, for example, on a virtual grid 603 on the examination table 106 can be used as a reliable reference. In some implementations, the clinician may use reference points 602, 604, or reference lines 606 on the examination table 106 to conveniently position a specific portion of the person 102 (e.g., a shoulder joint).

For example, as shown in FIG. 5, if the person 102 has identified the lesion 112 on the upper right thigh (see FIG. 1) as a lesion of interest to be tracked, the location of the lesion 112 can be defined as the displacement 608 (distance and direction) with respect to the reference plane 606. Note that the locating of a lesion of interest may not have to be extremely precise, if the measurement is accurate enough to distinguish the lesion of interest from adjacent lesions (e.g., within a centimeter or a few centimeters). The person 102 may need to remove clothing to fully expose the lesion of interest 112 during the scan.

In general, any kind of optical or other electromagnetic technology that is useful in locating a position of an object on a body can be used and the position of a reference point.

The location of the lesion can be expressed in distance along two or three orthogonal dimensions from the reference location. The distances can be expressed from an identified place on the lesion, for example, a point on one edge, or the “geographical center” or the intersection of two selected axes of the lesion. The location of the lesion can be stored on a general purpose computer 116 that performs the computation of location. In addition to the location and orientation of any lesion, the clinician is enabled, through the user interface, for example, to identify or indicate a lesion as being of interest that should be observed again in the future. This information can become part of the metadata that is stored in the memory device for use during a later examination.

FIG. 3 shows that digital photos of patient skin lesions can be taken by a scanner 114 that may include a portable memory card, such that the image information and related metadata can be automatically saved directly to the card either from the scanner or indirectly at the computer.

As suggested earlier, in some implementations, the camera 114 can provide a controlled image-taking environment to generate an RGB image of a skin lesion or a mole that is reproducible and accurate in terms of color, shape, and scale (dimensions) of the mole. This makes it easier to compare two images taken at two different times using typical software-oriented image comparison techniques to analyze, document, and report changes in skin lesions. This comparison and reporting can be done automatically in a subsequent scanning session, once the clinician indicates that a lesion previously identified as of interest is being scanned again.

In some implementations, the camera 114 can be a portable handheld RGB digital camera having a built-in memory, or alternatively connecting with an external memory device 304, e.g., a portable memory card. The camera 114 may also have a sensor, such as a CCD sensor, that acquires digital images in at least three channels: red, green, and blue. When features that appear in digital images, for example in digital images produced by a CMOS image sensor, are to be to quantitatively analyzed (for example, in medical applications), it is useful to reduce or remove the noise from the images before the analysis.

The location information, the identification of lesions of interest, and the image information of the lesion 302 can be used by a clinician to locate a particular lesion with certainty at a time after the scan has been done. For example, the information stored in memory 304 may determine the location of the lesion relative to the known reference point.

As shown in FIG. 4, software running on the computing device 308, allows the clinician to remind herself of lesions that seemed to need further observation, and to visualize the measurement parameter of the skin lesion 302 from prior skin scanning, perform analysis, and propose treatments.

The computing device 308 may include a display 310, a processor 312, and a memory 314. Software stored in the memory 314 performs a wide variety of functions. These functions can include controlling the scanner and the transceivers, fetching image information and other data from the scanner and transceivers, performing triangulation steps, providing a user interface that enables the clinician to identify lesions of interest, view lesions, and view changes in the lesions, analyzing successive images of a given lesion and providing automated information about changes in color, size, shape, and other characteristics of the lesion. In some implementations, the computing device 308 could be a handheld device, such as a personal digital assistant (PDA), which can be attached to the scanner 114 using a wired or wireless connection 316.

All the hardware components of the computing device 308 and the scanner 114, can in some examples be off-the-shelf components, that is, ready-made for a variety of uses and available for sale, lease, or license to the general public.

The computing device 308 may have access to other computers or server 318 through a network 316, such as the Internet. The computing device 308 may be connected to the network 316 by a variety of network connections such as a phone line, a cable or a wireless link.

The processor 312, under the control of the software, performs image analysis on the received image and provides the results of the analysis to display 310. Examples of the display 310 include cathode ray tube monitors, liquid crystal display monitors and touch-sensitive screens. Other displays may be used as well. If there is more than one lesion in the field-of-view of the camera, each lesion is analyzed separately. Alternatively, the lesion condition may be announced by an audible tone or visible indicator (for example, a light emitting diode) in which one tone or color indicator is used to denote the lesion does not need further evaluation (e.g., is benign) and a different tone or color indicator is used to specify that the lesion is in need of further evaluation.

Lesion information can be downloaded from the memory device 304 to a desktop or laptop computing device 308 at a different geographic location from where the lesion scanning is performed. Therefore, the location of the lesion and diagnosis notes of the clinician can be moved from place to place as needed to serve the interest of the patient, the current or future clinician, researchers, the party that supplies the scanner, and other entities.

As shown in FIG. 4, skin lesion image information acquired by the scanner 114 at time A and saved on the memory device 304 is displayed 401 for lesion analysis and evaluation along with an indication 403 of its location and orientation. Then, at a later time B, the same or a different clinician at the same or a different physical location can acquire image information 405 and 409 (and location information 407) that shows the lesion of interest for comparison. Multi-spectrally derived characteristics of the lesion of interest, such as relative lesion depth and volume, can also be obtained by analyzing the spectral properties of the skin lesion captured by the images (e.g., hemoglobin, melanin, bilirubin in skin). The results of automated mole mapping of a sequence of scans can also be displayed 407. The results of automated mole mapping can include images, overlaid images, analyzed images, and data, comparison data, analyzed data and other information.

Information associated with each skin scan may also include evaluation of the images by an expert in skin cancer, usually a dermatologist, a report to the patient and/or referring health practitioner including suspected diagnoses and recommendations for treatment of lesions of concern. By storing accurate numerical coordinates of a lesion that has been scanned, later work by the original clinician or by other clinicians may be simplified. When the scans are stored electronically and the coordinates of the lesion are also stored electronically, for example, a consulting clinician or a clinician who replaces the original one may be able to provide more effective services. Identification of the lesion on the patient can be faster and more accurate, for example.

Statistical data about the locations of lesions on a large number of patients might be used for purposes of analysis that is not easy or in some cases possible with data that is currently available.

In one specific example of the use of the system described here, a clinician first uses MelaFind to scan lesions of on the body of a patient. Each time the clinician takes an image of a lesion, the computer uses signals from the triangulation transceivers and the orientation sensors in the scanner automatically to determine the location and orientation of the lesion on the patient's body. The location and orientation information is stored as metadata with the image information on an electronic card that becomes part of the part of the patient's record. Also stored on the card (to become part of the patient's physical patient record) or on the computer are indications by the clinician of which lesions are of interest and ought to be tracked. That information can be entered by the clinician by a dedicated button on the scanner, or through a user interface of the computer that may display the scanned lesions on a diagram (or photograph) of the patient's body. Suppose that the patient moves to another city and a few months later visits a new clinician to have lesions scanned again. The patient's record, including the card, is transferred to the new clinician.

When the new clinician begins the scanning of the patient, she inserts the card into her own computer which then displays the locations of the prior scans and can show images and metadata associated with them, including the indication of the prior clinician of lesions that were of interest. The new clinician can then scan the lesions of interest. The computer then automatically performs comparisons of the initial scans with the new scans, determines changes in the color, size, shape, and other characteristics of the lesions and displays the change information to the clinician who can use it to improve the quality and speed, and reduce the cost, of providing services, such as mole mapping services to the patient.

Other implementations are also within the scope of the following claims.

1. A method comprising
in connection with scanning a lesion on the skin of a subject, automatically determining the location of the lesion on the subject,
storing the scan and the location on a portable memory device, and
including the memory device in a physical patient record of the subject for later use.

2. The method of claim 1 in which the scanning includes deriving image information corresponding to the lesion.

3. The method of claim 1 in which the scanning is performed in a manner that reduces the effects of ambient light on image information generated by the scan.

4. The method of claim 1 in which the location is determined using triangulation.

5. The method of claim 4 in which the triangulation comprises sending signals between transceivers located in fixed locations relative to the subject and a device located on or near and at a fixed location relative to the subject.

6. The method of claim 1 in which the portable memory device comprises flash memory.

7. The method of claim 1 also comprising automatically determining an orientation of the lesion on the subject.

8. The method of claim 1 also including scanning the lesion again at a later time.

9. The method of claim 1 in which the lesion comprises a pigmented skin lesion.

10. The method of claim 8 also comprising
automatically analyzing changes in the lesion represented by the scans of the lesion, and
reporting the changes to a clinician.

11. The method of claim 1 also including receiving an indication that the lesion is of interest to be later scanned for changes.

12. The method of claim 11 also including
at a later time identifying to a clinician that the lesion is of interest.

13. The method of claim 12 in which the identifying comprises a graphical indication on a depiction of the subject's body.

14. A method comprising in the presence of ambient light having a first set of characteristics, capturing first image information about a lesion on the skin of a subject using a scanner that screens the ambient light from the lesion,
later, in the presence of ambient light having a second set of characteristics, capturing second image information about the lesion using the scanner, and
by machine, analyzing the first image information and the second image information to infer changes in characteristics of the lesion.

15. The method of claim 14 in which the lesion comprises a pigmented skin lesion.

16. The method of claim 14 in which the machine analyzes changes in at least one of the size, color, or shape of the lesion.

17. The method of claim 14 in which the machine analyses changes in the lesion based on multi-spectral properties of the lesion.

18. The method of claim 14 also including displaying the results of the analysis to a clinician.

19. The method of claim 14 in which the image information is in an RGB format.

20. A method comprising
receiving image scans of a pigmented skin lesion of interest on a patient at two different times during which at least one of the color, size, shape, or non-visible multi-spectral characteristics of the pigmented skin lesion may have changed,
by machine performing an analysis of the changes of the pigmented skin lesion, and
reporting the result of the analysis to a clinician through a user interface.

21. A method comprising
storing on a portable memory device two images of a pigmented skin lesion acquired at two different times,
storing on the portable memory device information about the location of the pigmented skin lesion on the patient,
storing on the portable memory device an indication that that pigmented skin lesion is of interest, and
displaying the images, location information, and indication of interest information to a clinician in connection with a pigmented skin lesion mapping procedure.
